SEQUENCE LISTING (6 sequences)

SEQ ID NO: 1 - DNA, 19 bp, artificial sequence
  PCR Primer
ttggagctggtggcgtagg

SEQ ID NO: 2 - DNA, 19 bp, artificial sequence
  PCR Primer
  misc_feature (10)..(10): n is the 2′-O-tertButyl-dimethyl-silyl derivative of the guanosinribonucleotide.
ttggagctgntggcgtagg

SEQ ID NO: 3 - DNA, 19 bp, artificial sequence
  PCR primer
  misc_feature (9)..(10): n is the 2′-O-tertButyl-dimethyl-silyl derivative of the guanosinribonucleotide.
ttggagctnntggcgtagg

SEQ ID NO: 4 - DNA, 19 bp, artificial sequence
  PCR Primer
  misc_feature (8)..(8): n is the 2′-O-tertButyl-dimethyl-silyl derivative of the uridinribonucleotide.
  misc_feature (9)..(10): n is the 2′-O-tertButyl-dimethyl-silyl derivative of the guanosinribonucleotide.
ttggagcnnntggcgtagg

SEQ ID NO: 5 - DNA, 19 bp, artificial sequence
  PCR Primer
  misc_feature (7)..(7): n is the cytosine arabinoside.
  misc_feature (8)..(8): n is the 2′-O-tertButyl-dimethyl-silyl derivative of the uridinribonucleotide.
  misc_feature (9)..(10): n is the 2′-O-tertButyl-dimethyl-silyl derivative of the guanosinribonucleotide.
ttggagnnnntggcgtagg

SEQ ID NO: 6 - DNA, 19 bp, artificial sequence
  PCR Primer
ttggagctgttggcgtagg

(19) United States
(12) Patent Application Publication
(10) Pub. No.: US 2007/0082343 A1
(43) Pub. Date: Apr. 12, 2007

(54) Method for the control of segment-wise enzymatic duplication of nucleic acids via incomplete complementary strands

(76) Inventors: Peter Bendzko, Berlin (DE); Stephen Heymann, Berlin (DE); Hans Joos, Berlin (DE); Beate Kraffert, Berlin (DE); Ralf Bergmann, Dresden (DE); Matthias Leiser, Solingen (DE)

Correspondence Address: BUCHANAN INGERSOLL & ROONEY PC, P.O. BOX 1404, ALEXANDRIA, VA, 22313-1404, US

(21) Appl. No.: 11/358,576

(22) Filed: Feb. 21, 2006

(30) Foreign Application Priority Data
Oct. 7, 2005 (DE) 10 2005048503.0

(51) Int. Cl. C12Q 1/68; C12P 19/34

(52) U.S. Cl. 435/6; 435/91.2

(57) ABSTRACT

The invention relates to a new method for the control of enzymatic duplication of nucleic acids by the section via incomplete complementary strands. Fields of application of the invention are research, medical practice, gene-based analytics of biotechnological, agricultural and foodstuff products as well as criminology.

### CROSS REFERENCE TO RELATED APPLICATIONS

This application claims priority to pending German Patent Application No. 10 2005 048 503.0, filed on Oct. 7, 2005, and incorporated by reference herein.

### REFERENCE TO SEQUENCE LISTING

A sequence listing, in paper, is appended hereto and incorporated by reference herein.

### BACKGROUND OF THE INVENTION

DNA analytics have continuously gained in importance in the course of the last decade for research and medical practice and are increasingly penetrating other areas of human occupation. As representatives of the requirement and the increasing repertoire of DNA analysis techniques, figures from human genetics are quoted here: in the year of 2002, tests for about 750 polymorphous loci in the human genome were offered (EPF 2002)¹, whereas approx. 1,870 such detection kit and service offers are currently available (OMIM)². On account of approved causality and thus their meaningfulness, allele diagnostics for monogeneic hereditary diseases form the largest group with the highest turnover. On the other hand, finding combinations of hereditary predispositions and somatic mutations in the case of complex pathologies or susceptibilities for the same is naturally much more difficult: there is currently no superset of correlated DNA variants which together completely cover the vast majority of corresponding cases of illness and additionally make satisfactory therapy recommendations possible. The diagnostic requirements of the future will essentially differ from the current state of the art with regard to their complexity (quantity and nature of the aberrations to be detected simultaneously), sensitivity (extremely small mutant/wild type ratio) and robustness.
¹Ernst Peter Fischer, Das Genom, Fischer Taschenbuch Verlag, Reihe Fischer Kompakt, Frankfurt am Main, 2002, ISBN 3-596-15362-x

²OMIM: Online Mendelian Inheritance in Men, http://www.ncbi.nlm.nih.gov/entrez/guery.fcgi?db=OMIM

This situation also applies analogously to method and process-orientated areas of DNA recombinant technique and also the biochemical and fine chemical industry: cost and time-saving innovations are firing worldwide competition. Innovations in this regard make it possible, for example, to establish high throughput methods for gene cloning tasks.

The new and further development of universal methods of gene, genome and gene expression research likewise entails the complex of RNA analytics, including splicing and maturation processes of the mRNA, RNA-based enzymes and bioactive tools of expression regulation right down to first therapeutics.

In order to portray the richness of facets of the current state of art approximately adequately, the scope of a number of standard works of modern molecular biology were necessary. Molecular biology can still rightfully be termed the generator of methods engineering. Reference is made here to the textbooks by Seyffert³ and Lewin⁴. The extract relevant to the invention is shown below by portraying three typical basic cases. To a certain extent, many established techniques have recourse to these basic cases:
³Wilhelm Seyffert (Hrsg), Lehrbuch der Genetik, mit Beitr. von Rudi Balling u.a., 2. Aufl.-Heidelberg; Berlin: Spektrum, Akad. Verl., 2003, ISBN 3-8274-1022-3

⁴Benjamin Lewin, Genes VIII, Prentice Hall, 2004, ISBN: 0 1314 3981 2

Basic Case 1

Below, there is concern with the detection of a somatic nucleotide exchange relevant for disease etiology. We encounter it in a number of monogen-specific diagnostic tasks, predominantly in oncology, and it also returns in more complex questions:

Given an isolated DNA of a biological source, comprising k haplogenome equivalents. We know of the reference sequence of the chromosomal DNA fragment in question, S. Let sequence S be sufficiently unique in the given isolate. The position and nature (e.g. A to C) of the exchange in question in S are known in advance. The mutated DNA fragment S′ can be strongly underrepresented, but need not be.

The customary methods of distinguishing wild type and mutant essentially make use of mismatch effects of synthetic primers, substrates, labels and probes on enzyme and hybridisation reactions and the selective cleavability of sequences with restrictases, which are used in sequential assay steps. Various nucleotide derivatives and possibilities of biochemical conversion are used in numerous versions, which are developed, as a rule, to ensure patentability. They can be classified as follows:
1. increase of the number of copies by amplification of S and S′ with the aid of polymerases (PCR)⁵ and ligases (LCR)⁶,
   ⁵Mullis K, Faloona F, Scharf S, Saiki R, Horn G, Erlich H., Specific enzymatic amplification of DNA in vitro: the polymerase chain reaction, Cold Spring Harb Symp Quant Biol. 1986; 51 Pt 1:263-73
   ⁶Barany F., The ligase chain reaction in a PCR world, PCR Methods Appl. 1991 August; 1(1):5-16
2. position-specific incorporation of labelled and/or terminating substrate analogues (sequencing technique acc. to Sanger⁷, pyrosequencing⁸),
   ⁷Sanger F, Nicklen S, Coulson A R, DNA sequencing with chain-terminating inhibitors, Proc Natl Acad Sci USA. 1977 December; 74(12):5463-7
   ⁸M. Ronaghi, M. Uhlén and P. Nyrén, A sequencing method based on real-time pyrophosphate. Science 281 (1998), p. 363
3. template-dependent ligation of flush half-probes (OLA) or circularization (isothermal RCA or—vice versa—padlocking)⁹,
   ⁹Coutelle C., New DNA-analysis techniques, Biomed Biochim Acta. 1991; 50(1):3-10. (minireview)
4. beacon and scorpion-based techniques¹⁰,
   ¹⁰Leone G, van Schijndel H, van Gemen B, Kramer F R, Schoen C D, Molecular beacon probes combined with amplification by NASBA enable homogeneous, real-time detection of RNA, Nucleic Acids Res. 1998 May 1; 26(9):2150-5
5. primary or secondary (reaction product) immobilisation on solid phases (chip technologies)¹¹,
   ¹¹Gilles P N, Wu D J, Foster C B, Dillon P J, Chanock S J., Single nucleotide polymorphic discrimination by an electronic dot blot assay on semiconductor microchips, Nat Biotechnol. 1999 April; 17(4):365-70
6. single strand refolding (SSCP¹², dHPLC¹³).
   ¹²Orita M, Iwahana H, Kanazawa H, Hayashi K, Sekiya T., Detection of polymorphisms of human DNA by gel electrophoresis as single-strand conformation polymorphisms, Proc Natl Acad Sci USA. 1989 April; 86(8):2766-70
   ¹³
7. use of the varying melting temperature of double helix and heteroduplices of the same sequence (DNA/RNA, DNA/DNA* with *=structurally stiffened sugar [locked DNA] or substituted sugar phosphate backbone [PNA])¹⁴
   ¹⁴Petersen M, Wengel J., LNA: a versatile tool for therapeutics and genomics, Trends Biotechnol. 2003 February; 21(2):74-81. Review

The nucleotide derivatives fulfil the function of substrates of the polymerases or ligases, of primers of the polymerase chain reaction and of probes in the assay systems. In detail, the rough assignment results in the following picture:
1. A number of triphosphate analogues are accepted and incorporated by the polymerase as a substrate. In this way, labels (fluorescent, terminating, affine, (bio)chemically convertible or inert groups) and bonds (capable of hydrolysis or resistant to hydrolysis, B-form disturbances) are inserted by enzymatic condensation into the copies of either S or of S′, by which these can be discriminated.
2. Terminal and internal modifications in primers influence the selectability of S vs. S′ as a result of
   creation/removal of restriction sites;
   changes of the annealing temperature (mismatches);
   production of ligation ability (5′-p);
   cycle capability,
   enabling/exclusion of nucleolytic cleavability (proofreading, displacement, chimera cleavage).
3. Terminal modifications in probes are used for selective detection purposes on the basis of
   fluorescence quenchers/amplifiers;
   affine groups for immobilisation and for detection (Biotin, FITC).

Increasingly, a number of the aforementioned properties and purposes are being used in combination. For example Scorpions (Sigma-Aldrich) combine primer and probe function. In chip-based detection systems (Asper), substrate analogue incorporation is combined to form a technological sequence with the consequence of fragmentability, primer extension with synthesis stop and fluorescence marker incorporation. In specific cases of detection of mutated, cancer-relevant k ras minor components, either wild type cleavage and differential probe thermostability for sensitive detection of mutants via DNA-Elisa (Invitek GmbH) or PCR plus dHPLC (Nordiag SA) are implemented consecutively.

In systematic observation, it is conspicuous that amongst all the established combinations there has yet to be a reference in literature or a protective right for the utilisation of nucleotide modifications for the control of polymerase activity via the template. It is merely known that thymidine dimers (structural distortions caused under the influence of UV radiation by cyclo-addition of neighbouring deoxythymidines) only permit the incorporation of one dA with subsequent termination of the synthesis.

The question arises of whether a primer which is 100% complementary to an SNP location can simultaneously fulfill the purpose of effectively inhibiting the reverse-strand synthesis in a PCR mixture with the result of a decelerated or even no exponential amplification.

Basic Case 2

Direct Cloning Methods for PCR Products

Customary cloning methods for amplified dsDNA fragments are based either

1. conventionally on the use of auxiliary cloning sites attached as oligonucleotide extensions in the design of the primer, of subsequent restriction digestion of the PCR product and insertion into a correspondingly prepared vector¹⁵
   ¹⁵Normally, the attached auxiliary cloning material comprises the corresponding recognition sequence of the selected restrictase plus 3-4 arbitrary nucleotides, which are needed in order to secure the full endonucleolytic effect of the restriction enzyme. This method has the severe disadvantage that the ancillary cloning locations in the PCR product must be unique, otherwise the amplificate is fragmented. The uniqueness is a problem, in particular with long amplificates of unknown sequence. or
2. on the T/A complementarity of protruding 3′-ends of insert and vector¹⁶ or
   ¹⁶Non-proofreading polymerases attach 1-3 additional nucleotides to the 3′ end of a freshly synthesised complementary strands, depending on the nucleotide transferases. The rule of thumb is: more than 90% of the 3′ ends have one supernatant nucleotide, which is 85% dA; a second nucleotide (likewise mainly dA) is found in about 1% of the synthesis products, a third one in 0.01%. Thermo-stable polymerases, manifesting a higher template tolerance compared with RNA dependent on the ions (Mn²⁺/Mg²⁺), have a stronger tendency to product extension with additional nucleotides according to experience. This explains why there is absolutely unsatisfactory success in including raw PCR products into a vector with blunt ends. Partially, 3′-T-tailed vectors are of assistance here.
3. on the ligase activity of the topoisomerase I, which inserts a blunt or 3′-dA tailed PCR product into a specifically prepared commercial cloning vector ^{17, 18, 19, 20, 21, 22, 23} extremely quickly.
   ¹⁷Fundamental and further-reaching explanations on this time and expenditure saving method as well as its applications for expression, see inter alia the following 6 quotes.
   ¹⁸Shuman, S. (1994) J. Biol. Chem. 269: 32678-32684
   ¹⁹Clark, J. M. (1988) Nuc. Acids Res. 16: 9677-9678
   ²⁰Mead, D. et al. (1991) Bio/Techniques 9: 657-663
   ²¹Bernard, P. and Couturier, M. (1992) J. Mol. Biol. 226: 735-745
   ²²Bernard, P. et al. (1993) J. Mol. Biol. 234: 534-541
   ²³Rand, K. N. (1996) Elsevier Trends Technical Tips Online

Here, the underlying idea is generating a PCR product fraction which is given the vector-complementary ends as soon as it originates.

The cleaved vector has two groups of four indents. Accordingly, the amplification primers are provided with the four complementary bases on their 5′ ends, e.g. the forward primer with the inner four bases AATT of the EcoR I recognition sequence, the return primer with the inner four bases TCGA of the Hind III recognition sequence.

The modification will only be inserted with one fraction (about 1/10 to 1/100) of the primer, by which on the one hand normal exponential amplification of the fragment is to take place between the primers. The ‘normal’ amplificates withdraw from cloning because of non-complementarity of their ends to the vector. The amplificate descendants generated by occasional incorporation of stop variants of the primers have the required cohesive ends.

Basic Case 3

Due to alternative splicing, numerous genes provide more than one mature mRNA and the respective amino acid sequence. The accumulated distribution statistics of the number of transcripts and exons per gene as a function of the gene length proven experimentally and bio-computationally (by EST alignment) is permanently updated for human genes (Ensembl V.32 ff)²⁴.
²⁴S. http://www.ensembl.org

In practice, it is repeatedly seen that a number of mRNAs are erroneously considered to be the canonic transcript of the gene in question. In fact, however, they are a tumour-associated splice forms of the given gene (Xu and Lee 2003)²⁵. The reason is that in the past, i.e. before the human genome project was started, cDNA cloning and sequencing was the method of choice for research of gene expression in human tissues, for which surgically removed tumour material was frequently used.
²⁵XU, Q. and Lee, C. (2003). Discovery of novel splice forms and functional analysis of cancer-specific alternative splicing in human expressed sequences. Nucleic Acids Res. 31, 5635-5643

In alternative splicing, the following stereotypes basically permanently recur: exon skipping, multiple exon skipping, mutually exclusive exon skipping, cryptic exons, facultative promoters, splice signal attenuation, intron retention, cascade splice processes—also combinations of the aforementioned types.

In a wide-scale machine experimental study (Hiller et al. 2004)²⁶ it was shown that a much larger number of putative splice forms can be functionally significant for many genes. The molecular “fine tuning” of the splicing process is known to be tissue-dependent, mutation, condition and context sensitive. The splice form simulation in a computer abstracts from these many degrees of freedom and thus opens a path for collecting biological arguments for the existence of further splice forms which can then be searched for strait-forward, especially in the case of extreme minor components which are under-represented in cDNA and EST libraries by probabilistic reasons and therefore are found even more rarely than they are represented in the mRNA pool of a biological source anyway. This analysis is thus of predictive value.
²⁶M. Hiller, R. Backofen, S. Heymann, A. Busch, T. M. GläBer and J.-C. Freytag Efficient prediction of alternative splice forms using protein domain homology, In Silico Biology 4, 0017, 2004

The verification of the predicted alternative splice forms is customarily done in laboratory experiments via Northern blots, or bioinformatically as far as there are highly homologous sequences in the sequence data available; failure to find such homologues far and away does not prove a splice form as non-existing. The data situation is still too incomplete for this purpose. So there is a considerable requirement for further improvement.

The invention is based on the task of developing a robust and quick method for control of segment-wise enzymatic duplication of nucleic acids, used inter alia for the analysis of alternative splice forms, in the detection of nucleic acid sequence variants and for gene cloning.

The invented is implemented according to the main claim, the subclaims are preferential variants.

### BRIEF SUMMARY OF THE INVENTION

The invention relates to a new method for the control of enzymatic duplication of nucleic acids by the section via incomplete complementary strands. Fields of application of the invention are research, medical practice, gene-based analytics of biotechnological, agricultural and foodstuff products as well as criminology.

### BRIEF DESCRIPTION OF THE SEVERAL VIEWS OF THE DRAWINGS

FIG. 1: Partial inhibitory according to the invention “Method for the control of segment-wise enzymatic duplication of nucleic acids via incomplete complementary strands”. The amount of amplification product in use of a synthetic oligonucleotide with a stop function (bottom row) has been reduced compared to the control (top row).

FIG. 2: Principal portrayal of the application of the method according to the invention to the proof of alternative splice forms. Explanations in the text.

FIG. 3: Typical electrophoresis image for basic case 1 of the method for the control of enzymatic duplication of nucleic acid by the section via incomplete complementary strands. Details in the text.

### DETAILED DESCRIPTION OF THE INVENTION

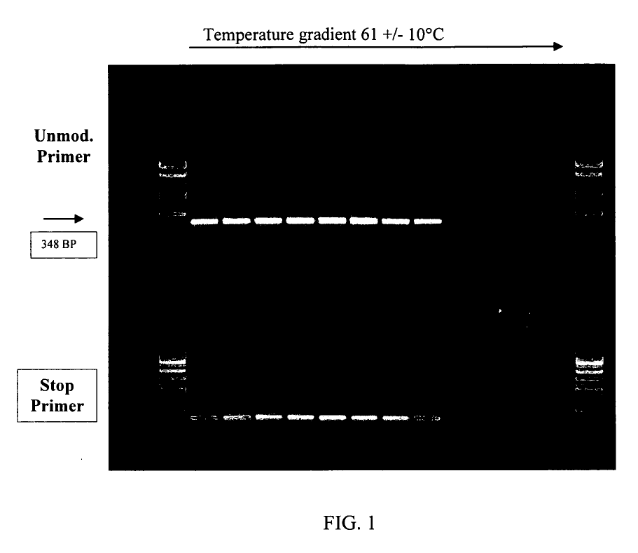

(1) In molecular biology routine experiments of the inventors, so called chimeric oligonucleotides of the form 5′ NₖXNₗ3′ are used in a completely different connection and purpose than that of the invention. In 5′ NₖXNₗ3′ N stands for an arbitrary one of the natural deoxyribonucleotides dA, dC, dG, dT, X for an arbitrary one of the natural ribonucleotides A, C, G, T; k and l is the monomer figures of the deoxyribonucleotide residues in the synthetic chimerical oligonucleotide. Surprisingly, a drastic reduction of the exponential DNA amplification was observed if the 2′ protective group of the ribonucleotide X had erroneously not been removed. This unexpected inhibitory effect has been shown in FIG. 1.

The following chemical group was used as a 2′ stop function:

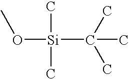

The inhibitory effect was shown to be reproducible in repeat experiments and finally led to the underlying idea of the invention.

(2) It is seen that the inhibitory effect according to the invention tended to become stronger, the more ribonucleotide components with 2′ protective group were incorporated into chimeric oligonucleotides of the same length and sequence, e.g. 5′ Nₖ₋₁X₂Nₗ3′,

5′ Nₖ₋₂X₃Nₗ3′, 5′ Nₖ₋₃X₄Nₗ3′ etc. In even-numbered ribonucleotide components with 2′ protective group, the inhibitory effect even proved to be quantitative (see embodiment 1). Thus, the foundation for the subtle control of the segment-wise enzymatic duplication of nucleic acids has been laid. In fact, the fine analysis of the control intervention into the process of selective duplication of DNA fragments according to the invention showed no impairment of the first strand synthesis on the DNA template in the presence of a chimeric oligonucleotide primer with protected ribonucleotides. Stoppage of the complementary strand synthesis in the following cycle of the PCR reaction was recognised as the reason for inhibitory effect observed. Thus, the resulting incomplete complementary strand cannot serve as a template for subsequent strand syntheses in all the following cycles because the part section Nₗ of the chimeric oligonucleotide is too short for primer binding at the given annealing temperature. The outcome is a partial inhibition or even complete prevention of the exponential DNA amplification. Both effects—partial and total inhibition—are of decisive importance for various applications of the invention to basic cases 1 to 3, to which attention shall be paid further below.

(3) In the further elaboration of the invention, other bulky attachments in 2′ position of the ribonucleotides within chimeric oligonucleotides were also found suitable for producing the control effect described under (2), e.g. 2′-O triisopropylsilyl groups, 2′-O alkyl groups and others.

(4) Nucleotide components are known to bear further reactive groups which can be changed in chemical and biochemical conversion processes. The effects according to the invention described under (2) also occur to various extent if a) the natural sugars ribose or desoxyribose are replaced by arabinose, b) the natural phosphodiester bonds are changed by modifications making them stable to hydrolysis, c) the sugar-phosphate backbone of the synthetic oligonucleotides is replaced by inter-base bridges of other chemical nature to ensure stop effects on complementary strand synthesis, d) chemical changes are made to the nitrogen bases, which, although they do not impair the formation of the complementary base pairs A-T and G-C, are not accepted/tolerated by the polymerase as a readable template, e) additional chemical bonds exist between adjacent components of the synthetic oligonucleotide which result in structure faults and f) mixed changes of types (2), (3) and (4a-e) in the synthetic oligonucleotide.

(5) The following facts are common to all embodiments of the present invention under (1) to (4): The principle of controlling the segment-wise enzymatic nucleic acid duplication via incomplete complementary strands was unknown or unnoticed prior to this invention. This type of control is based on the stop effect of special synthetic oligonucleotides on the polymerase chain reaction. It is neither the result of a changed primer function nor of a substrate, probe or clamp effect. It is solely based on the double role in the start of the forward reaction, by which a synthetic oligonucleotide of the property described above becomes an integral part of the nucleic acid copies, and in the stoppage of the subsequent synthesis of complementary strands. This is the most important demarcation criterion of the invention and makes it unambiguously distinguishable from all customary embodiments and special applications of the polymerase chain reaction for detection, cloning and discrimination purposes of nucleic acids.

(6) From (1) to (5), it can automatically be deduced that absolutely no complete DNA double helices result or complete double helices form in the mixture with defined incomplete ones in the control of enzymatic duplication of nucleic acid by the section via incomplete complementary strands according to the invention. This demarcates the invention against the customary methods of enzymatic synthesis of nucleic acid copies with stochastic length distribution as a result of synthesis termination by means of dideoxyribonucleotide triphosphates (Sanger method). The stoppage of synthesis in the method according to the invention is done at a defined, stated position. This for its part makes it possible to generate nucleic acid copies with cohesive ends without the customary efforts of restriction cleavage of auxiliary cloning sequences, which can then be ligated into correspondingly prepared cloning vectors or recombinant constructs. In this way, considerable progress is achieved compared with the repertoire of customary cloning techniques (cf. basic case 2).

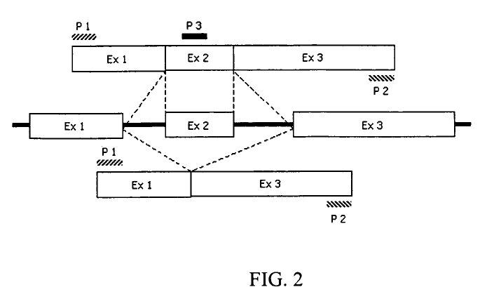

(7) The use of the oligonucleotides with a stop function according to the invention makes selective amplification of alternative splice forms possible for the first time. This best becomes clear from the following FIG. 2. The diagram exemplarily illustrates the mode of procedure on an arbitrary tri-exonic gene, of which it is postulated, for the sake of clarity, that its long splice form (the so-called holoform, top) originates by inclusion of all three exons into the mature mRNA, whereas the short splice form comes about by skipping of the middle exon in the extreme stoichiometric deficiency (bottom).

Selection and use of the primers are as follows: the two splice forms are transcribed into cDNA_from the total RNA with the 3′ primer (P2) and amplified with the primer pair P1/P2. From the electrophoretic image, we only expect one band for the long splice form; according to requirements, the short one is a minor component and, if at all, visible as a shadow.

But if we additionally mix the exon-2 specific stop primer (P3) to the PCR mixture, the exponential duplication of the long splice form is removed and, after a corresponding adaptation of the number of cycles, the amplificate of the short splice form results (cf. basic case 3).

Benefits:

With Regard to Basic Case 1
Replacement of the customary multi-step process by a patent-free single-step method by omission
   of the nested PCR steps;
   of the restriction cleavage;
   of the DNA-Elisa
      with its own class of DNA based detection assays of lower error-proneness.
Less laborious procedures and cost-intensive accessories (enzymes, streptavidin plates, conjugate)
If applicable, higher sensitivity (and thus even earlier cancer recognition)
Backward compatibility (possibility of applying the Elisa or SSCP used up to then)
Applicability in the sense of mass screening and for pre-selection
Possibility of automation

With Regard to Basic Case 2
Omission of the restriction cleavage step of amplificates at flanking auxiliary cloning sites
Doing without the risk of undesired fragmenting in long amplificates of unknown sequence
Doing without being dependent on purchase of prefabricated vectors

With Regard to Basic Case 3
Process integration of bio-information forecasts of alternative splice forms and purposeful experimental verification of their existence in the tissue examined
Avoidance of noxious substances (formamide) by doing without conventional Northern Blot procedures
Being able to find minor component splice forms

### EMBODIMENTS

1. Basic case 1 is realised according to the invention by the following mixture: In parallel PCR mixtures, each of 25 μl reaction volume, 50 ng of isolated genomic DNA from SW620 cells (a human epithelial-like cell with deposit no. ATCC CCL 227) are subjected to amplification in an Eppendorf Mastercycler gradient according to the following programme: 94° C./5′ for preliminary denaturation of the genomic template; 94° C./30″ for the denaturation step in each cycle; 61±10° C./30″ for annealing of 12 identical reaction aliquots in each case at a variable temperature in the range from 51 to 71° C. with a constant temperature increase or cooling rate of 3° C. per second; 72° C./1′ for primer elongation; 35 cycles, 72° C./5′ for the final synthesis completion; cooling to 4° C. for keeping up to the gel-electrophoretic analysis. To ensure identical reactant concentrations, work was done with a master mix. It contained 0.76×PCR standard buffer, 1.5 mM MgCl₂, 1.5 μg/ml BSA; 76 μM of each of the four desoxyribonucleotide triphosphates, 0.5 units of taq polymerase relative to each mixture aliquot; 15 pmol of the common backward primer 5′ TACCCTCTCACGAAACTCTG 3′ (SEQ ID NO:1) relative to each mixture aliquot. This primer is specific for one section in exon 1 of the human k-ras gene. Five part amounts of the master mix, sufficient for 12 mixture aliquots each, were separately mixed with 15 pmol of the forward primer listed below relative to each mixture aliquot, the sequence of which is identical and specific for the genomic range around codon 12/13 of the human k-ras gene. Together, the forward and backward primer cover 348 base pairs on the genomic DNA:
1.1 5′ ttggagctggtggcgtagg 3′ (SEQ ID NO:2), specific for the k-ras wild type allel in SW620;
1.2 5′ TTGGAGCTGG*TGGCGTAGG 3′(SEQ ID NO:3), specific for the k-ras wild type allel in SW620, G* means the 2′-O-tertButyl-dimethyl-silyl derivative of the guanosinribonucleotide;
1.3 5′ ttggagctg*G*TGGCGTAGG 3′(SEQ ID NO:4), specific for the k-ras wild type allel in SW620, G* means the 2′-O-tertButyl-dimethyl-silyl derivative of the guanosinribonucleotide;
1.4 5′ TTGGAGCU*G*G*TGGCGTAGG 3′(SEQ ID NO:5), specific for the k-ras wild type allel in SW620, G* means the 2′-O-tertButyl-dimethyl-silyl derivative of the guanosinribonucleotide and U* the 2′-O-tertButyl-dimethyl-silyl derivate of the uridinribonucleotide;
1.5 5′ TTGGAGC*U*G*G*TGGCGTAGG 3′(SEQ ID NO:6), specific for the k-ras wild type allel in SW620, G* means the 2′-O-tertButyl-dimethyl-silyl derivate of the guanosinribonucleotide, U* the 2′-O-tertButyl-dimethyl-silyl derivative of the uridinribonucleotide and C* the cytosine arabinoside.

A sixth identical part quantity of the master mix, likewise sufficient for 12 mixture aliquots, was mixed with a forward primer (again 15 pmol relative to each mixture aliquot), which is specific in the same genomic position in the k-ras gene as primers 1.1 to 1.5, but unlike them is specific for the chromosome in SW620, which manifests a glycine to valine mutation (G12V) in codon 12 of the k-ras gene.
1.6 5′ TTGGAGCTGTTGGCGTAGG 3′ (SEQ ID NO:7), specific for the somatically muted (G12V) DNA in SW620.

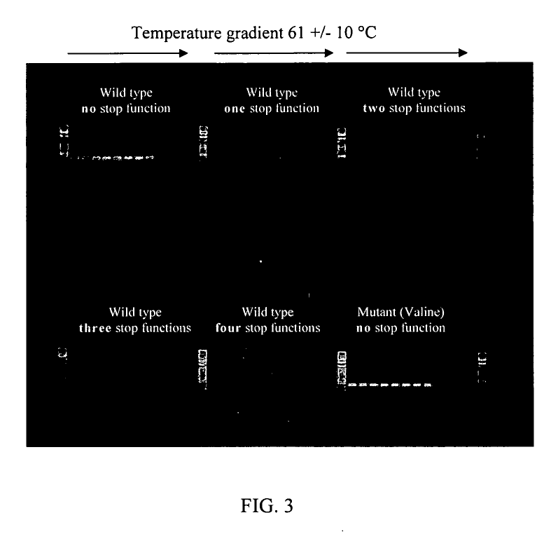

After the PCR, 6 μl of each reaction mixture was mixed in the customary way with Orange G in glycerine/H₂0 and applied to a horizontal agarose gel (1.5% in TAE buffer plus 0.1 μg/ml ethidium bromide). The order of application is as follows: top row, left to right—series 1.1, 1.2, 1.3; bottom row, left to right—series 1.4, 1.5, 1.6, each delimited by a DNA standard with fragments of known lengths. A constant voltage of 7V/cm is applied to the electrophoresis gel in submarine mode. The separation lasted 45 minutes. A photograph of this gel under UV light can be seen in FIG. 3.

Observation on FIG. 3:

Quantitative inhibition is observed in even-numbered series of stop functions (2 and 4), whereas odd-numbered series (1 and 3) cause a partial inhibition. The intensity of the wild-type and mutant amplificates from parallel mixtures with primers without any stop function (top left and bottom right) is used to compare the quantities.

1. Method for the control of segment-wise enzymatic duplication of nucleic acid via incomplete complementary strands, wherein the in vitro synthesis of DNA double helices is stopped before its completion without the use of terminating substrate analogues,
with this stop of the polymerase activity during the complementary strand synthesis being caused by a suppression of the continuous readability of the template at pre-determined spots in the template and
with this suppression of the continuous readability of the template at pre-determined spots in the template being caused by the use of synthetic oligonucleotides with integrated nucleotide monomers of an unnatural chemical property.

2. Method according to claim 1, wherein the unnatural chemical property of the integrated nucleotide monomers does not impair the base pairing ability to complementary nucleic acid sections.

3. Method according to claim 1, wherein the unnatural chemical property of the integrated nucleotide monomers does not impair the primer extension effect of the synthetic oligonucleotides for polymerase reactions.

4. Method according to claim 1, wherein the unnatural nucleotide monomers in the number of synthetic oligonucleotides contains derivatives of known per se sugars.

5. Method according to claim 1, wherein the unnatural nucleotide monomers in the number of synthetic oligonucleotides contain hydrolysis-resistant inter-sugar-bonds.

6. Method according to claim 1, wherein certain monomers along the synthetic oligonucleotides are not connected via phosphodiester bonds.

7. Method according to claim 1, wherein the unnatural nucleotide monomers along synthetic oligonucleotides have undergone additional bonds to adjacent nucleotides.

8. Method according to claim 1, wherein the unnatural nucleotide monomers in along synthetic oligonucleotides contain additional functional groups on the bases capable of pairing.

9. Method according to claim 1, wherein the synthetic oligonucleotides contain additional bonds between two or two each adjacent nucleotide monomers.

10. Method according to claim 9, wherein the additional bonds are produced by simultaneous incorporation of neighbouring bases cyclo-added in advance.

11. Method according to claim 9, wherein the additional bonds are subsequently produced by cyclo-addition of the bases.

12. Method according to claim 4, wherein the various sugars are pentoses or derivatives of pentoses.

13. Method according to claim 12, wherein modified deoxyriboses or modified riboses are used as pentoses.

14. Method according to claim 12, wherein arabinose is used as a pentose.

15. Method according to claim 1, wherein the modified riboses contain bulky additional groups on the 2′-carbon atom.

16. Method according to claim 15, wherein the bulky additional groups on the 2′-carbon atom are selected from the group consisting of O-linked triisopropylsilyl groups, O-linked tertButyl-dimethylsilyl groups, and O-linked alkyl groups.

17. (canceled)

18. (canceled)

19. Method according to claim 1, wherein the synthetic oligonucleotides are mixed polymers of natural desoxyribonucleotides and the designated derivates.

20. Method according to claim 1, wherein the synthetic oligonucleotides contain one of more nucleotide derivative(s) of the designated types.

21. Method according to claim 20, wherein position and succession of the designated nucleotide derivatives in the synthetic oligonucleotide are derived from its sequence.

22. Method according to claim 1, further including at least one step selected from the group consisting of using the synthetic oligonucleotides for the detection of nucleic acid sequence variations, using the synthetic oligonucleotides for the detection of nucleic acid minor components in complex mixtures, using the synthetic oligonucleotides for the production of DNA molecules with defined single and double strand sections, using the synthetic oligonucleotides for the production of DNA molecules with protruding single-stranded ends, using the synthetic oligonucleotides for the production of DNA molecules entering into affine bonds with mobile or stationary reactants, using the synthetic oligonucleotides for the production of DNA molecules entering into chemical bonds with mobile or stationary reactants, using wherein the synthetic oligonucleotides for the production of labelled DNA molecules, and using the synthetic oligonucleotides for the detection of alternative splice forms.

23. (canceled)

24. (canceled)

25. (canceled)

26. (canceled)

27. (canceled)

28. (canceled)

29. (canceled)
